(19) United States
(12) Patent Application Publication
(10) Pub. No.: US 2026/0102128 A1
(43) Pub. Date: Apr. 16, 2026

(54) METHODS AND SYSTEMS FOR USING HYPERSPHERICAL HARMONICS FOR RADIOTHERAPY TREATMENT PLANNING AND DELIVERY TASKS

(71) Applicant: VANDERBILT UNIVERSITY, Nashville, TN (US)

(72) Inventor: Adam D. Yock, Nashville, TN (US)

(21) Appl. No.: 19/360,446

(22) Filed: Oct. 16, 2025

Related U.S. Application Data
(60) Provisional application No. 63/708,025, filed on Oct. 16, 2024.

(51) Int. Cl. A61B 6/03; A61B 6/00; A61N 5/10

(52) U.S. Cl. CPC A61B 6/03; A61B 6/5294; A61N 5/1039

(57) ABSTRACT

An example system includes a medical imaging device, a server, a computing device, and a display. The server can be configured to convert images and/or any other medical data from the medical imaging device into Hyperspherical Harmonic representations to compress, index, search, process, and retrieve the medical data. The computing device can be configured to access the images, medical data, and Hyperspherical Harmonic representations stored on the server to improve radiotherapy and other forms of medical treatment.

### CROSS-REFERENCE TO RELATED APPLICATIONS

This application claims the benefit of U.S. provisional Ser. No. 63/708,025, filed on Oct. 16, 2024, and titled “METHODS AND SYSTEMS FOR USING HYPERSPHERICAL HARMONICS FOR RADIOTHERAPY TREATMENT PLANNING AND DELIVERY TASKS” the disclosure of which is expressly incorporated herein by reference in its entirety.

### BACKGROUND

Spherical harmonics provide a robust mathematical framework for analyzing 3D data, making them highly relevant to medical imaging techniques like MRI and CT scans, which rely on three-dimensional data reconstruction. By applying spherical harmonics, the accuracy and efficiency of reconstructing complex anatomical structures in medical images can be significantly enhanced. This approach improves image resolution, reduces computational complexity, and allows for more precise visualization and diagnosis.

Hyperspherical Harmonics (HSH) are a high-dimensional generalization of spherical harmonics. HSH have not been used to represent medical images or reconstructions. It may be desirable to use HSH for medical imaging, image reconstruction, and radiotherapy planning.

### SUMMARY

In some aspects, the techniques described herein relate to a computer-implemented method including: determining a set of (n−1)-dimensional sample points in a spherical coordinate system; calculating, at a respective coordinate of each of the set of (n−1)-dimensional sample points, respective values of individual n-dimensional Hyperspherical Harmonic (HSH) basis functions up through a given maximum order; converting n-dimensional medical data into an HSH representation; and constructing an approximation of the n-dimensional medical data from the HSH representation.

In some aspects, the step of converting the n-dimensional medical data into the HSH representation includes representing the n-dimensional medical data using HSH by finding a set of coefficients that, when used to combine individual n-dimensional HSH basis functions in a weighted sum, minimize an error of the resulting value at each of the set of (n−1)-dimensional sample points compared to the true value of the n-dimensional medical data at each of the set of (n−1)-dimensional sample points.

In some aspects, the step of constructing the approximation of the n-dimensional medical data from the HSH representation includes using the HSH representation as a series of coefficients to calculate a weighted sum of the respective values of individual n-dimensional HSH basis functions at each of the set of (n−1)-dimensional sample points, resulting in the approximation of the n-dimensional medical data.

In some aspects, the n-dimensional medical data includes a 4D Computed Tomography (CT) image, a 4D Magnetic Resonance Imaging (MRI) image, a 4D Positron Emission Tomography (PET) image, a 4D binary map of one or more anatomic structures, a 4D parametric map of one or more anatomic structures, a 4D radiation dose distribution from an external beam radiotherapy plan, a 4D radiation dose distribution from a brachytherapy plan, a 4D radiation dose distribution from one or more brachytherapy sources, a 5D multi-energy Computed Tomography (CT) image, a 5D multi-energy Cone-Beam Computed Tomography (CBCT) image, a 6D map of radiological depth through an image subject, a 4D radiation intensity distribution from a series of coplanar external beam radiotherapy plan radiation source positions, or a 5D radiation intensity distribution from a series of non-coplanar external beam radiotherapy plan radiation source positions.

In some aspects, the method further includes estimating a 4D radiation dose distribution corresponding to a radiotherapy plan for a given anatomic configuration using the HSH representation of the n-dimensional medical data.

In some aspects, the method further includes aligning the n-dimensional medical data by minimizing the value of a distance metric calculated between the HSH representation of the n-dimensional medical data and respective HSH representations of one or more different n-dimensional medical data sets.

In some aspects, the method further includes estimating radiation intensity distribution corresponding to a radiation dose distribution for a given radiotherapy plan using the HSH representation of the n-dimensional medical data.

In some aspects, the method further includes optimizing the given radiotherapy plan.

In some aspects, the techniques described herein relate to a computer-implemented method for performing a radiotherapy task including: receiving a first HSH representation of first n-dimensional medical data; receiving a second HSH representation of second n-dimensional medical data; inputting the first HSH representation and the second HSH representation into a trained machine learning model; and predicting, using the trained machine learning model, a third HSH representation of third n-dimensional medical data.

In some aspects, the method further includes constructing an approximation of the third n-dimensional medical data from the third HSH representation.

In some aspects, the method further includes converting the first n-dimensional medical data into the first HSH representation, and converting the second n-dimensional medical data into the second HSH representation.

In some aspects, the radiotherapy task is calculating the radiation dose distribution that corresponds to a given anatomic configuration and radiation intensity distribution, the first n-dimensional medical data is an anatomic configuration as depicted in a medical image, the second n-dimensional medical data is a radiation intensity distribution, and the third n-dimensional medical data is a radiation dose distribution.

In some aspects, the radiotherapy task is determining the boundaries of an anatomic structure, the first n-dimensional medical data is an anatomic configuration as depicted in a medical image, the second n-dimensional medical data is an indicator of an anatomic structure of interest, and the third n-dimensional medical data is a binary map or parametric map of the anatomic structure of interest.

In some aspects, the radiotherapy task is reconstructing a medical image from a detected transmitted radiation signal, the first n-dimensional medical data is a radiation intensity distribution, the second n-dimensional medical data is a detected transmitted radiation signal, and the third n-dimensional medical data is an anatomic configuration as depicted in the medical image.

In some aspects, the medical image is a Computed Tomography (CT) image, a Cone-Beam Computed Tomography (CBCT) image, a multi-energy Computed Tomography (CT) image, or a multi-energy Cone-Beam Computed Tomography (CBCT) image.

In some aspects, the techniques described herein include a system including a medical imaging device; a server operably coupled to the medical imaging device, where the server is configured to: receive a medical image from the medical imaging device; convert the medical image into an hypespherical harmonic (HSH) representation by: determining a set of (n−1)-dimensional sample points in a spherical coordinate system; calculating, at a respective coordinate of each of the set of (n−1)-dimensional sample points, respective values of individual n-dimensional HSH basis functions up through a given maximum order; converting n-dimensional medical data into an HSH representation; and constructing an approximation of the n-dimensional medical data from the HSH representation; a computing device in operable communication with the server by a network; and a display operably coupled to the computing device; wherein the computing device is configured to: retrieve, from the server, the HSH representation; and output a depiction of the HSH representation for display to a user; and in response to a user input, retrieve from the server, the medical image; and output a depiction of the medical image for display.

In some aspects, the computing device is further configured to: optimize a radiotherapy procedure based on the HSH representation.

In some aspects, the computing device is further configured to receive a search query from the user, and, based on the search query, retrieve the HSH representation from the server.

In some aspects, the medical imaging device includes a computed tomography (CT) system.

In some aspects, the server is further configured to receive a plurality of medical images from the medical imaging device, convert the plurality of medical images into a corresponding plurality of HSH representations, and index the plurality of HSH representations for search or retrieval of the corresponding plurality of medical images.

It should be understood that the above-described subject matter may also be implemented as a computer-controlled apparatus, a computer process, a computing system, or an article of manufacture, such as a computer-readable storage medium.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF THE DRAWINGS

The components in the drawings are not necessarily to scale relative to each other. Like reference numerals designate corresponding parts throughout the several views.

FIG. 1 is a flowchart of an example method for representing n-dimensional medical data with Hyperspherical Harmonics (HSH) according to an implementation described herein.

FIG. 2A is a diagram of how the value of a data point in a second HSH-based feature space can be approximated by comparing it and its value in a first HSH-based feature space with those of other pairs of data points through interpolation or other operations using relative similarity or distance metrics.

FIG. 2B is a block diagram of a trained machine learning model used for performing a radiotherapy task according to an implementation described herein.

FIG. 3 is a flowchart of an example method for performing a radiotherapy task according to an implementation described herein.

FIG. 4 is an example computing device.

FIG. 5 is an example computing system configured to use HSH representations to improve the retrieval and use of medical images, according to an example implementation described herein.

FIG. 6 is an example comparison of an original CT image and dose distribution with Hyperspherical Harmonic representations for an example patient.

FIGS. 7A-7D illustrate example comparisons of methods of representing computed tomography data and dose date, where FIG. 7A illustrates head and neck CT data, FIG. 7B illustrates Prostate CT data, FIG. 7C illustrates head and neck dose data, and FIG. 7D illustrates prostate dose data.

FIG. 8A illustrates 4D Hyperspherical Harmonics used to model a femoral head as a point cloud.

FIG. 8B illustrates 4D Hyperspherical Harmonics used to model five anatomical structures as point clouds.

### DETAILED DESCRIPTION

Unless defined otherwise, all technical and scientific terms used herein have the same meaning as commonly understood by one of ordinary skill in the art. Methods and materials similar or equivalent to those described herein can be used in the practice or testing of the present disclosure. As used in the specification, and in the appended claims, the singular forms “a,” “an,” “the” include plural referents unless the context clearly dictates otherwise. The term “comprising” and variations thereof as used herein is used synonymously with the term “including” and variations thereof and are open, non-limiting terms. The terms “optional” or “optionally” used herein mean that the subsequently described feature, event or circumstance may or may not occur, and that the description includes instances where said feature, event or circumstance occurs and instances where it does not. Ranges may be expressed herein as from “about” one particular value, and/or to “about” another particular value. When such a range is expressed, an aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

As used herein, the terms “about” or “approximately” when referring to a measurable value such as an amount, a percentage, and the like, is meant to encompass variations of ±20%, ±10%, ±5%, or ±1% from the measurable value.

### Definitions

As used herein, Hyperspherical Harmonics (HSH) are a high-dimensional generalization of spherical harmonics, typically defined on the surface of a sphere in a multidimensional space. They are mathematical functions that form an orthonormal basis for functions defined on the surface of a hypersphere (an n-dimensional generalization of a circle or sphere). In a 3D space, a sphere is the set of all points equidistant from a center point. In higher dimensions, the analogous concept is a hypersphere. For example, a 4D hypersphere would be the set of points equidistant from a center in four-dimensional space. Spherical harmonics in 3D describe the angular part of the solutions to Laplace's equation, which appears in many areas of physics like quantum mechanics, electromagnetism, and gravitational problems. In higher dimensions, HSH serve a similar role, but they work for spaces with more than three dimensions.

The term “artificial intelligence” is defined herein to include any technique that enables one or more computing devices or comping systems (i.e., a machine) to mimic human intelligence. Artificial intelligence (AI) includes, but is not limited to, knowledge bases, machine learning, representation learning, and deep learning. The term “machine learning” is defined herein to be a subset of AI that enables a machine to acquire knowledge by extracting patterns from raw data. Machine learning techniques include, but are not limited to, logistic regression, support vector machines (SVMs), decision trees, Naïve Bayes classifiers, and artificial neural networks. The term “representation learning” is defined herein to be a subset of machine learning that enables a machine to automatically discover representations needed for feature detection, prediction, or classification from raw data. Representation learning techniques include, but are not limited to, autoencoders. The term “deep learning” is defined herein to be a subset of machine learning that that enables a machine to automatically discover representations needed for feature detection, prediction, classification, etc. using layers of processing. Deep learning techniques include, but are not limited to, artificial neural network or multilayer perceptron (MLP).

Machine learning models include supervised, semi-supervised, and unsupervised learning models. In a supervised learning model, the model learns a function that maps an input (also known as feature or features) to an output (also known as target or targets) during training with a labeled data set (or dataset). In an unsupervised learning model, the model learns patterns (e.g., structure, distribution, etc.) within an unlabeled data set. In a semi-supervised model, the model learns a function that maps an input (also known as feature or features) to an output (also known as target or target) during training with both labeled and unlabeled data.

As used herein, the dimensionality of medical data (e.g. n-dimensional medical data) refers to the number of variables or coordinates required to describe a point in the image space. Each additional dimension provides more information, contributing to the way the data is captured, processed, and visualized. For example, a three-dimensional (3D) image (x, y, z) captures spatial information in three dimensions: the width (x), height (y), and depth (z) of an object. Additionally, a four-dimensional (4D) image (x, y, z, value) can capture spatial information in three dimensions and a non-spatial value (i.e. “value”). The non-spatial value may represent various types of information, such as intensity, density, or another measurable property, which adds detailed information to the spatial structure, enriching the image data. This is the standard for most medical imaging techniques like CT or MRI scans, which capture volumetric data values at a set of 3D coordinates. Additionally, a five-dimensional (5D) image can capture, for example, spatial information in three dimensions and two non-spatial values (x, y, z, value1, value2), or spatial information in four dimensions and one non-spatial value (x, y, z, w, value).

### Example Methods and Systems

Implementations of the present disclosure include improvements to compression, digital image processing, and simulations, for example. Medical imaging generates huge amounts of data. The amount of data is compounded by treatment data. For example, in conventional a radiation therapy procedure, a CT scan can be used that includes a 3D voxel map, and then radiation fluence can be mapped on to the CT scan to generate a model that includes both structural information and radiation dosage information for each voxel in the CT scan. This creates “high dimensional” data where the 3D structural information is layered with additional data.

Applying analytical and machine learning techniques to large high dimensional datasets can be computationally prohibitive. As the resolution of the data grows, the number of voxels in each scan also increases, further limiting voxel-based techniques. Machine learning based techniques, for example, often assume a fixed and limited input size. Moreover, higher resolutions can require more complex machine learning based techniques, and higher resolution training data. Thus, compute, memory, model design and data requirements can be significant limitations to voxel-based techniques.

Implementations of the present disclosure overcome the limitations of voxel-based techniques for representing high dimensional medical data by applying Hyperspherical Harmonics (HSH) to represent medical data with any number of dimensions (referred to herein as “n-dimensional” data). HSH techniques described herein allow for extremely effective lossy compression of radiotherapy data (e.g., dosage maps). Some implementations of the present disclosure can enable lossy compression to file sizes of 5% or less than the original file size, less 2% or less than the original file size, or 1% or less than the original file size. This allows, for example, a conventional 250 MB file to be compressed to about 12.5 or 2.5 MB, allowing for efficient use of machine learning or other techniques on the data.

Despite the significant size reductions achievable by implementations of the present disclosure, the compressed file sizes can still include sufficient information for physics-based or machine learning techniques to be applied to the data for effective radiotherapy planning. The HSH techniques described herein can be extremely effective at preserving essential data required for radiotherapy planning while removing data that is not necessary. For example, the compressed images described herein can be used either to plan radiotherapy procedures, or as part of a computer system that optimizes or searches for a best radiotherapy procedure by evaluating many different options. Thus, HSH as described herein is an improvement over conventional systems used for compression of such data like Spherical and Zernike coordinate systems because these techniques are less suitable for high dimensional data used in radiotherapy and other medical imaging contexts.

Additionally, the compression techniques described herein can be used to improve the speed of search, navigation, indexing, and display of medical imaging data. For example, transmitting a 250 MB conventional image over a network, or retrieving it from a hard disk, can take multiple seconds, introducing inefficiencies in data review of many images. Likewise, techniques for searching images, finding similar images, etc. can have execution times that scale with the amount of data in the image files. Thus, the present disclosure contemplates computer systems that use HSH-compressed images for search, navigation, indexing, and display, while preserving ground-truth images for archival purposes, or for performing steps in the radiotherapy planning procedure.

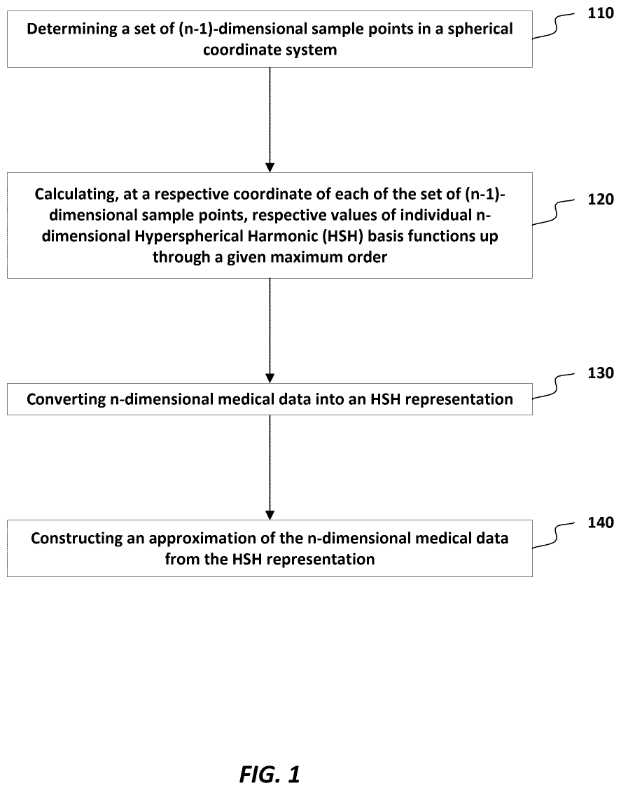

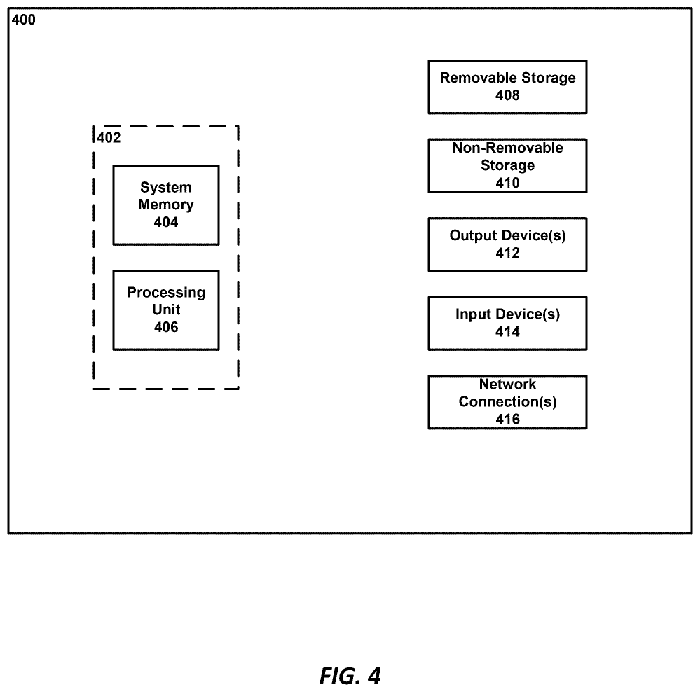

Referring now to FIG. 1, a flowchart of an example method for representing n-dimensional medical data with Hyperspherical Harmonics is described. This disclosure contemplates that the operations shown in FIG. 1 can be implemented using one or more computing devices such as the computing devices shown in FIG. 4 (e.g. at least one processor and memory). One technical advantage of representing medical data using HSH is that the HSH formalism represents data more efficiently and compactly (e.g., by a factor of 10-100×). Among other things, this may result in a smaller file size and lower computer memory and processing resources required to manipulate such n-dimensional medical data.

In some implementations, the n-dimensional medical data includes a 4D Computed Tomography (CT) image, a 4D Magnetic Resonance Imaging (MRI) image, a 4D Positron Emission Tomography (PET) image, a 4D binary map of one or more anatomic structures, a 4D parametric map of one or more anatomic structures, a 4D radiation dose distribution from an external beam radiotherapy plan, a 4D radiation dose distribution from a brachytherapy plan, a 4D radiation dose distribution from one or more brachytherapy sources, a 5D multi-energy Computed Tomography (CT) image, a 5D multi-energy Cone-Beam Computed Tomography (CBCT) image, a 6D map of radiological depth through an image subject, a 4D radiation intensity distribution from a series of coplanar external beam radiotherapy plan radiation source positions, or a 5D radiation intensity distribution from a series of non-coplanar external beam radiotherapy plan radiation source positions. It should be understood that the n-dimensional medical data above are only provided as examples. This disclosure contemplates that the n-dimensional medical data can be other than those provided as examples. Additionally, it should be understood that the dimensionality of 4D images such as CT, MRI, PET include data coordinates (x, y, z) and value and that the dimensionality of 5D images such as multi-energy CT, CBCT include data coordinates (x, y, z), energy level, and value.

At step 110, the method includes determining a set of (n−1)-dimensional sample points in a spherical coordinate system.

At step 120, the method includes calculating, at a respective coordinate of each of the set of (n−1)-dimensional sample points, respective values of individual n-dimensional HSH basis functions up through a given maximum order.

At step 130, the method includes converting n-dimensional medical data into an HSH representation. For example, the step of converting the n-dimensional medical data into the HSH representation includes representing the n-dimensional medical data using HSH by finding a set of coefficients that, when used to combine individual n-dimensional HSH basis functions in a weighted sum, minimize an error of the resulting value at each of the set of (n−1)-dimensional sample points compared to the true value of the n-dimensional medical data at each of the set of (n−1)-dimensional sample points. The HSH coefficients can be considered the “features” used for machine learning operations, and thus implementations of the present disclosure allow for feature engineering which is not possible when applying conventional deep learning techniques to conventional medical images.

At step 140, the method includes constructing an approximation of the n-dimensional medical data from the HSH representation. For example, the step of constructing the approximation of the n-dimensional medical data from the HSH representation includes using the HSH representation as a series of coefficients to calculate a weighted sum of the respective values of individual n-dimensional HSH basis functions at each of the set of (n−1)-dimensional sample points, resulting in the approximation of the n-dimensional medical data.

In some implementations, the method further includes estimating a 4D radiation dose distribution corresponding to a radiotherapy plan for a given anatomic configuration using the HSH representation of the n-dimensional medical data.

In some implementations, the method further includes aligning the n-dimensional medical data by minimizing the value of a distance metric calculated between the HSH representation of the n-dimensional medical data and respective HSH representations of one or more different n-dimensional medical data sets.

In some implementations, the method further includes estimating radiation intensity distribution corresponding to a radiation dose distribution for a given radiotherapy plan using the HSH representation of the n-dimensional medical data. Optionally, the method further includes optimizing the given radiotherapy plan.

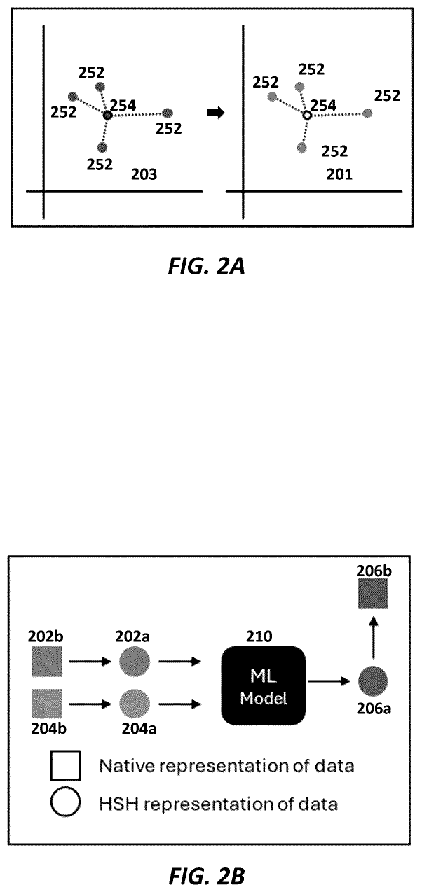

FIG. 2A illustrates the value of a data point in a second HSH-based feature space 201 can be approximated by comparing it and its value in a first HSH-based feature space 203 with those of other pairs of data points through interpolation or other operations using relative similarity or distance metrics. For example, data points 252 can represent medical data, including a treatment outcome, for a set of patients in the first HSH-based feature space 203 and the second HSH-based feature space 201. Interpolation techniques can be used to estimate a likely treatment outcome for a datapoint corresponding to a new patient's medical data 254, where the new patient's medical data 254 does not include the treatment outcome.

Referring now to FIG. 2B, a block diagram of a trained machine learning model 210 used for performing a radiotherapy task is shown. In FIG. 2B, the machine learning model 210 is operating in inference mode. The machine learning model 210 has therefore been trained with a data set (or “dataset”) and is configured to make predictions based on new input data.

Accordingly, such a model is sometimes referred to herein as a “trained machine learning model” or a “deployed machine learning model.” In some implementations, the machine learning model 210 is a supervised machine learning model. Supervised machine learning models include, but are not limited to, random forest classifiers, support vector machines, Naïve Bayes classifiers, and artificial neural networks. It should be understood that random forest classifiers, support vector machines, Naïve Bayes classifiers, and artificial neural networks are provided only as example supervised machine learning models. This disclosure contemplates that the trained machine learning model 210 can be other supervised learning models.

As described above, a supervised machine learning model “learns” a function that maps an input 202a, 204a (also known as feature or features) to an output 206a (also known as target or targets) during training with a labeled data set. Machine learning model training is discussed in further detail below. In some implementations, a trained supervised machine learning model is configured to classify the input 202a, 204a into one of a plurality of target categories (i.e., the output 206a). In other words, the trained model can be deployed as a classifier. In other implementations, a trained supervised machine learning model is configured to provide a probability of a target (i.e., the output 206a) based on the input 202a, 204a. In other words, the trained model can be deployed to perform a regression.

Optionally, in some implementations, the machine learning model 210 is an artificial neural network (ANN). An artificial neural network (ANN) is a computing system including a plurality of interconnected neurons (e.g., also referred to as “nodes”). This disclosure contemplates that the nodes can be implemented using a computing device (e.g., a processing unit and memory as described herein). The nodes can be arranged in a plurality of layers such as input layer, output layer, and optionally one or more hidden layers. An ANN having hidden layers can be referred to as deep neural network or multilayer perceptron (MLP). Each node is connected to one or more other nodes in the ANN. For example, each layer is made of a plurality of nodes, where each node is connected to all nodes in the previous layer. The nodes in a given layer are not interconnected with one another, i.e., the nodes in a given layer function independently of one another. As used herein, nodes in the input layer receive data from outside of the ANN, nodes in the hidden layer(s) modify the data between the input and output layers, and nodes in the output layer provide the results. Each node is configured to receive an input, implement an activation function (e.g., binary step, linear, sigmoid, tanH, or rectified linear unit (ReLU) function), and provide an output in accordance with the activation function. Additionally, each node is associated with a respective weight. ANNs are trained with a dataset to maximize or minimize an objective function. In some implementations, the objective function is a cost function, which is a measure of the ANN's performance (e.g., error such as L1 or L2 loss) during training, and the training algorithm tunes the node weights and/or bias to minimize the cost function. This disclosure contemplates that any algorithm that finds the maximum or minimum of the objective function can be used for training the ANN. Training algorithms for ANNs include, but are not limited to, backpropagation.

As shown in FIG. 2B, the machine learning model 210 is configured to provide output 206a based on the input 202a, 204a. In the examples described herein, the input 202a, 204a includes a first HSH representation input 202a of first n-dimensional medical data and a second HSH representation input 204a of second n-dimensional medical data, and the output 206a is a third HSH representation output 206a of third n-dimensional medical data. The machine learning model 210 is therefore trained to map the input 202a, 204a to the output 206a. In other words, the input 202a, 204a includes one or more “features” that are input into the machine learning model 210, which predicts the output 206a which is therefore the “target” of the machine learning model 210.

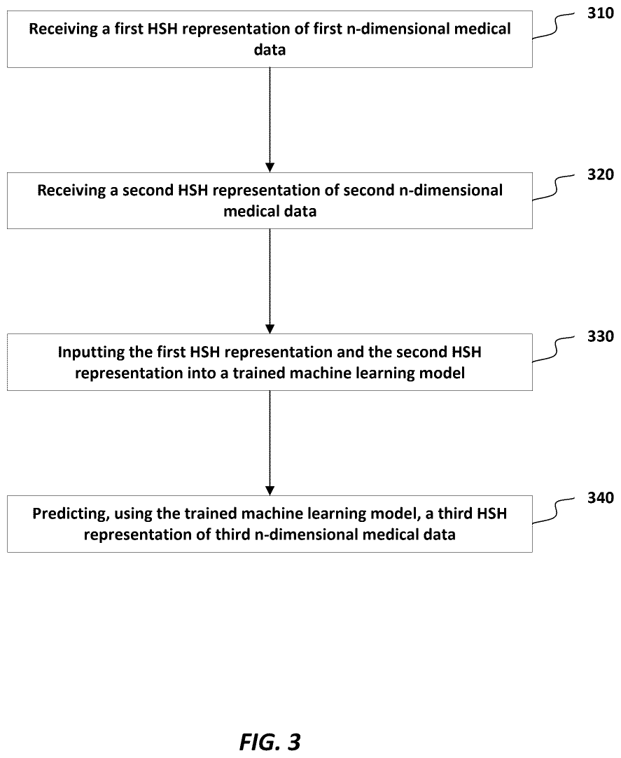

Referring now to FIG. 3, a flowchart of an example method for performing a radiotherapy task is described. This disclosure contemplates that the operations shown in FIG. 3 can be implemented using one or more computing devices such as the computing devices shown in FIG. 4 (e.g. at least one processor and memory). One technical advantage of performing machine learning on medical data represented using HSH is that the HSH formalism represents data more efficiently and compactly (initial data suggests by a factor of 10-100×). Among other things, this may result in a smaller file size and lower computer memory and processing resources required to manipulate such n-dimensional medical data.

At step 310, the method includes receiving a first HSH representation of first n-dimensional medical data. First n-dimensional medical data can be input 202b and its first HSH representation can be input 202a are illustrated in FIG. 2B. Example first n-dimensional medical data is described below for different implementations of the radiotherapy task. Optionally, the method further includes converting the first n-dimensional medical data into the first HSH representation. Conversion of n-dimensional medical data into an HSH representation is described above with regard to FIG. 1.

At step 320, the method includes receiving a second HSH representation of second n-dimensional medical data. Second n-dimensional medical data 204b and its second HSH representation input 204a are illustrated in FIG. 2B. Example second n-dimensional medical data is described below for different implementations of the radiotherapy task. Optionally, the method further includes converting the second n-dimensional medical data into the second HSH representation. Conversion of n-dimensional medical data into an HSH representation is described above with regard to FIG. 1.

At step 330, the method includes inputting the first HSH representation and the second HSH representation into a trained machine learning model. In some implementations, the machine learning model is a supervised machine learning model. An example supervised machine learning model is an ANN. Optionally, the ANN is a deep learning model. The trained machine learning model 210 is illustrated in FIG. 2B.

At step 340, the method includes predicting, using the trained machine learning model, a third HSH representation of third n-dimensional medical data. Optionally, the method further includes constructing an approximation of the third n-dimensional medical data from the third HSH representation. Construction of an approximation of n-dimensional medical data from an HSH representation is described above with regard to FIG. 1. Third n-dimensional medical data can be output 206b and its third HSH representation can be output 206a as illustrated in FIG. 2B.

In some implementations, the radiotherapy task is calculating the radiation dose distribution that corresponds to a given anatomic configuration and radiation intensity distribution, the first n-dimensional medical data is an anatomic configuration as depicted in a medical image, the second n-dimensional medical data is a radiation intensity distribution, and the third n-dimensional medical data is a radiation dose distribution.

In some implementations, the radiotherapy task is determining the boundaries of an anatomic structure, the first n-dimensional medical data is an anatomic configuration as depicted in a medical image, the second n-dimensional medical data is an indicator of an anatomic structure of interest, and the third n-dimensional medical data is a binary map or parametric map of the anatomic structure of interest.

In some implementations, the radiotherapy task is reconstructing a medical image from a detected transmitted radiation signal, the first n-dimensional medical data is a radiation intensity distribution, the second n-dimensional medical data is a detected transmitted radiation signal, and the third n-dimensional medical data is an anatomic configuration as depicted in the medical image. Optionally, the medical image is a Computed Tomography (CT) image, a Cone-Beam Computed Tomography (CBCT) image, a multi-energy Computed Tomography (CT) image, or a multi-energy Cone-Beam Computed Tomography (CBCT) image.

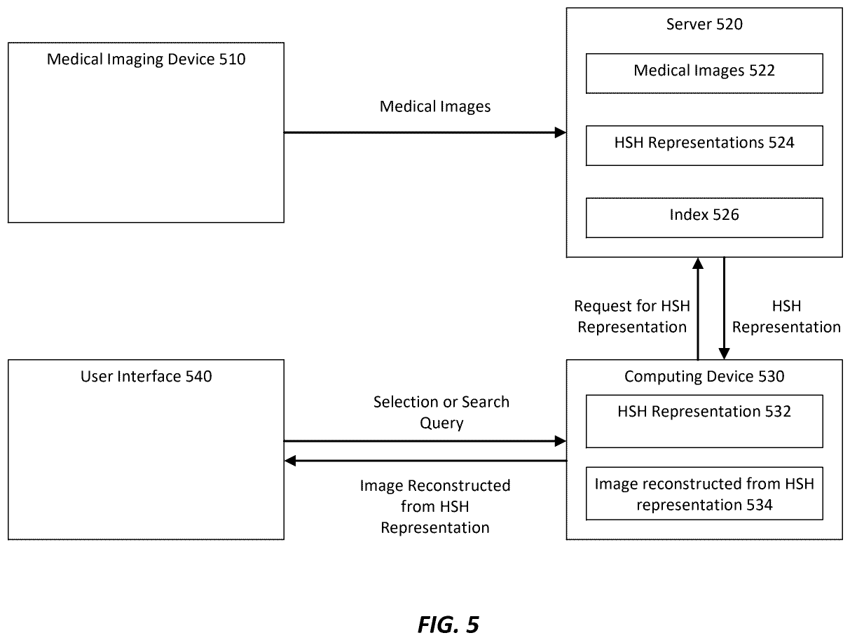

FIG. 5 illustrates an example system according to implementations of the present disclosure. The system can include a medical imaging device 510. The medical imaging device 510 can include any of the medical imaging devices described herein, including a CT scanner, a CBCT scanner, or a multi-energy CBCT scanner.

The medical imaging device 510 can be in operable communication with a server 520, for example using any combination of wired or wireless networks. Medical images (e.g., any of the medical images described herein), can be transmitted from the medical imaging device 510 to the server 520 and stored on the server 520 as medical images 522.

The server 520 can include one or more computing devices 400 as shown in FIG. 4. The server 520 can be configured to perform any of the methods described herein. For example, the server 520 can be configured to process the medical images 522 into corresponding HSH representations 524 of the medical images 522. The server 520 can use the HSH representations 524 to build an index 526 for efficient search and retrieval of the HSSH representations 524 and/or medical images 522 that correspond to the HSH representations.

A computing device 530 (or any number of computing devices) can be in operable communication with the server 520 to access the HSH representations 524 or corresponding medical images 522. The computing device 530 can retrieve, from the server, a subset of HSH representations 532 from the HSH representations 524, and optionally convert them into a reconstructed image 534 for display. Alternatively or additionally, the computing device 530 can be configured to enable a user to search the server 520 for HSH representations in the set of HSH representations 524 that are similar to a given HSH representations. Optionally, the searching can be accelerated using the index 526 of HSH representations.

The reconstructed image 534 can be output to a user interface 540, which can include one or more displays. The user interface 540 can also include user inputs enabling he user to cause the computing device 530 and/or server 520 to display different reconstructed images 534.

In an example workflow, a user can use the user interface 540 to view a set of reconstructed images 534 stored on the computing device based on HSH representations 532. The user can then select one or more of the reconstructed images 534 to cause the computing device 530 to retrieve the original medical image corresponding to the HSH representation that the reconstructed image was based on. This allows the computing device 530 to store and manipulate smaller files (the HSH representations 532 and reconstructed images 534) while the complete medical images 522 remain available for retrieval and use.

In another example workflow, the user can input a search into the user interface 540 to cause the computing device 530 to access the index 526 of HSH representations 524 to efficiently retrieve the HSH representation 524 of a particular image, the original medical image 522 corresponding to the HSH representation, or a reconstructed image 534 based on the HSH representation.

In some implementations, the computing device 530 is a computer configured for optimizing and/or planning a radiotherapy procedure. The optimizing and/or planning can optionally be performed using the HSH representation 532, reducing the computational complexity, memory, and processing time required to perform the steps on the computing device 530. For example, the computing device 530 can be a local “edge” device like a laptop, personal computer, tablet, etc. where processing power and/or memory are limited, preventing local analysis of the complete medical images 522 stored on the server 520. FIGS. 2A and 2B herein include example methods for using high dimensional HSH data to increase the performance of machine learning models and data comparisons, for example.

It should be appreciated that the logical operations described herein with respect to the various figures may be implemented (1) as a sequence of computer implemented acts or program modules (i.e., software) running on a computing device (e.g., the computing device described in FIG. 4), (2) as interconnected machine logic circuits or circuit modules (i.e., hardware) within the computing device and/or (3) a combination of software and hardware of the computing device. Thus, the logical operations discussed herein are not limited to any specific combination of hardware and software. The implementation is a matter of choice dependent on the performance and other requirements of the computing device. Accordingly, the logical operations described herein are referred to variously as operations, structural devices, acts, or modules. These operations, structural devices, acts and modules may be implemented in software, in firmware, in special purpose digital logic, and any combination thereof. It should also be appreciated that more or fewer operations may be performed than shown in the figures and described herein. These operations may also be performed in a different order than those described herein.

Referring to FIG. 4, an example computing device 400 upon which the methods described herein may be implemented is illustrated. It should be understood that the example computing device 400 is only one example of a suitable computing environment upon which the methods described herein may be implemented. Optionally, the computing device 400 can be a well-known computing system including, but not limited to, personal computers, servers, handheld or laptop devices, multiprocessor systems, microprocessor-based systems, network personal computers (PCs), minicomputers, mainframe computers, embedded systems, and/or distributed computing environments including a plurality of any of the above systems or devices. Distributed computing environments enable remote computing devices, which are connected to a communication network or other data transmission medium, to perform various tasks. In the distributed computing environment, the program modules, applications, and other data may be stored on local and/or remote computer storage media.

In its most basic configuration, computing device 400 typically includes at least one processing unit 406 and system memory 404. Depending on the exact configuration and type of computing device, system memory 404 may be volatile (such as random access memory (RAM)), non-volatile (such as read-only memory (ROM), flash memory, etc.), or some combination of the two. This most basic configuration is illustrated in FIG. 4 by box 402. The processing unit 406 may be a standard programmable processor that performs arithmetic and logic operations necessary for operation of the computing device 400. The computing device 400 may also include a bus or other communication mechanism for communicating information among various components of the computing device 400.

Computing device 400 may have additional features/functionality. For example, computing device 400 may include additional storage such as removable storage 408 and non-removable storage 410 including, but not limited to, magnetic or optical disks or tapes. Computing device 400 may also contain network connection(s) 416 that allow the device to communicate with other devices. Computing device 400 may also have input device(s) 414 such as a keyboard, mouse, touch screen, etc. Output device(s) 412 such as a display, speakers, printer, etc. may also be included. The additional devices may be connected to the bus in order to facilitate communication of data among the components of the computing device 400. All these devices are well known in the art and need not be discussed at length here.

The processing unit 406 may be configured to execute program code encoded in tangible, computer-readable media. Tangible, computer-readable media refers to any media that is capable of providing data that causes the computing device 400 (i.e., a machine) to operate in a particular fashion. Various computer-readable media may be utilized to provide instructions to the processing unit 406 for execution. Example tangible, computer-readable media may include, but is not limited to, volatile media, non-volatile media, removable media and non-removable media implemented in any method or technology for storage of information such as computer readable instructions, data structures, program modules or other data. System memory 404, removable storage 408, and non-removable storage 410 are all examples of tangible, computer storage media. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In an example implementation, the processing unit 406 may execute program code stored in the system memory 404. For example, the bus may carry data to the system memory 404, from which the processing unit 406 receives and executes instructions. The data received by the system memory 404 may optionally be stored on the removable storage 408 or the non-removable storage 410 before or after execution by the processing unit 406.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high level procedural or object-oriented programming language to communicate with a computer system. However, the program(s) can be implemented in assembly or machine language, if desired. In any case, the language may be a compiled or interpreted language and it may be combined with hardware implementations.

The following numbered clauses define some non-limiting example embodiments according to aspects described herein.

Clause 1: A method of representing n-dimensional (sample coordinate+value) medical data with Hyperspherical Harmonics comprising:
Determine a set of (n−1)-dimensional sample points in a spherical coordinate system. At each sample point, the data to be modeled has a single value.

At the coordinate of each sample point, calculate the values of individual n-dimensional Hyperspherical Harmonic (HSH) basis functions up through a given maximum order.

To convert n-dimensional data into an HSH representation: Represent the data using HSH by finding the set of coefficients that, when used to combine individual HSH basis functions in a weighted sum, minimize the error of the resulting value at each sample point compared to the true value of the data at that point, for example, by using Least Squares Optimization.

To construct n-dimensional data from an HSH representation: Use the HSH representation as a series of coefficients to calculate a weighted sum of the values of individual HSH basis functions at each sample point, resulting in an approximation of the original data.

Clause 2: The method of Clause 1 where the n-dimensional data is a 4D Computed Tomography (CT) image.

Clause 3: The method of Clause 1 where the n-dimensional data is a 4D Cone-Beam Computed Tomography (CBCT) image.

Clause 4: The method of Clause 1 where the n-dimensional data is a 4D Magnetic Resonance Imaging (MRI) image.

Clause 5: The method of Clause 1 where the n-dimensional data is a 4D Positron Emission Tomography (PET) image.

Clause 6: The method of Clause 1 where the n-dimensional data is a 4D binary map of one or more anatomic structure(s).

Clause 7: The method of Clause 1 where the n-dimensional data is a 4D parametric map of one or more anatomic structure(s).

Clause 8: The method of Clause 1 where the n-dimensional data is a 4D radiation dose distribution from an external beam radiotherapy plan.

Clause 9: The method of Clause 1 where the n-dimensional data is a 4D radiation dose distribution from a brachytherapy plan.

Clause 10: The method of Clause 1 where the n-dimensional data is a 4D radiation dose distribution from one or more brachytherapy source(s).

Clause 11: The method of Clause 1 where the n-dimensional data is a 5D multi-energy Computed Tomography (CT) image.

Clause 12: The method of Clause 1 where the n-dimensional data is a 5D multi-energy Cone-Beam Computed Tomography (CBCT) image.

Clause 13: The method of Clause 1 where the n-dimensional data is a 6D map of radiological depth through the image subject.

Clause 14: The method of Clause 1 where the n-dimensional data is a 4D radiation intensity distribution from a series of coplanar external beam radiotherapy plan radiation source positions.

Clause 15: The method of Clause 1 where the n-dimensional data is a 5D radiation intensity distribution from a series of non-coplanar external beam radiotherapy plan radiation source positions.

Clause 16: A method of aligning two or more n-dimensional medical data sets by minimizing the value of a distance metric calculated between their HSH representations. For example, minimizing the Euclidean distance between points whose coordinates correspond to the coefficients that weigh individual HSH basis functions in an HSH representation of the data.

Clause 17: A method of estimating the 4D radiation dose distribution corresponding to a radiotherapy plan for a given anatomic configuration by representing an n-dimensional image (e.g. CT or MRI) as HSH coefficients, identifying one or more similar anatomic configuration(s) by minimizing the value of a distance metric calculated between their HSH representations, and using this similarity to infer the radiation dose distribution of the anatomic configuration of interest by considering the dose distribution(s) of the similar anatomic configuration(s) (e.g. by interpolation or a weighted average).

Clause 18: A method of estimating the radiation intensity distribution corresponding to a radiation dose distribution for a given radiotherapy plan by representing the radiation dose distribution as HSH coefficients, identifying one or more similar radiation dose distribution(s) by minimizing the value of a distance metric calculated between their HSH representations, and using this similarity to infer the radiation intensity distribution of the radiation dose distribution of interest by considering the radiation intensity distribution(s) of the similar radiation dose distribution(s) (e.g. by interpolation or a weighted average).

Clause 19: A method of optimizing a radiotherapy plan that uses the method of Clause 17 to estimate the 4D radiation dose distribution based on an anatomic configuration and then the method of Clause 18 to estimate the radiation intensity distribution based on the estimated 4D radiation dose distribution, and lastly determines treatment machine parameters to approximate the estimated radiation intensity distribution.

Clause 20: A method of performing a radiotherapy Task X by determining the HSH representation of an Output C from HSH representations of Inputs A and B using an artificial neural network. The artificial neural network is trained by optimizing a set of hyperparameters that result in a model that most accurately predicts the HSH representations of C from those of A and B when applied to a pre-existing training set of corresponding A, B, and C data. The trained neural network is then used to infer an unknown HSH representation of C corresponding to known HSH representations of A and B.

Clause 21: The method of Clause 20 where Task X is calculating the radiation dose distribution that corresponds to a given radiation intensity distribution such as that which characterizes a radiotherapy plan, inputs A and B are an anatomic configuration (input A) as depicted in a medical image and a radiation intensity distribution (input B), and output C is a radiation dose distribution.

Clause 22: A method of optimizing the radiation intensity distribution such as that which characterizes a radiotherapy plan by iteratively calculating the radiation dose distribution that results from the combination of a radiation intensity distribution in conjunction with an anatomic configuration (as in Clause 21), evaluating the radiation dose distribution based on clinical metrics, and selecting the radiation intensity distribution that results in the most desirable radiation dose distribution per the clinical metrics, and then determining treatment machine parameters to best approximate the desired radiation intensity distribution.

Clause 23: The method of Clause 20 where Task X is determining the boundaries of an anatomic structure, inputs A and B are an anatomic configuration (input A) as depicted in a medical image and an indicator of the anatomic structure of interest (input B), and output C is a binary map or parametric map of the anatomic structure of interest.

Clause 24: The method of Clause 20 where Task X is reconstructing a medical image from a detected transmitted radiation signal, inputs A and B are a radiation intensity distribution (input A) and a detected transmitted radiation signal (input B), and output C is an anatomic configuration as depicted in a medical image.

Clause 25: The method of Clause 24 where the medical image is a Computed Tomography (CT) image.

Clause 26: The method of Clause 24 where the medical image is a Cone-Beam Computed Tomography (CBCT) image.

Clause 27: The method of Clause 24 where the medical image is a multi-energy Computed Tomography (CT) image.

Clause 28: The method of Clause 24 where the medical image is a multi-energy Cone-Beam Computed Tomography (CBCT) image.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for.

### Example 1

One implementation disclosed herein is methods of representing radiotherapy data using the particular formalism of Hyperspherical Harmonics (HSH). Also disclosed are systems that use the data in this format to perform tasks for radiotherapy treatment planning and delivery.

This use of HSH can be considered similar to the concept of Fourier Analysis performed in spherical coordinates of dimensions 4 or higher.

This mathematical formalism can be applied to 3D point cloud data or to data represented in a 3D matrix of voxels. Data represented in greater than 3D can similarly be considered as the mathematics generalize to higher dimensions trivially.

In radiotherapy, it is therefore applicable to datasets such as:
1) Volumetric imaging (e.g. CTs and MRIs),
2) Radiation dose distribution maps,
3) Structure contours (described as a point cloud of vertices or as a voxelized map),
4) Maps of radiation intensity that compose a radiotherapy treatment plan,
5) Additional data structures derived from the above (e.g. a 6D representation of radiological depth throughout the patient anatomy).

The coincidence that the geometry and symmetry of the HSH functions are similar to those observed naturally in these radiotherapy datasets allows for the HSH formalism to represent the data more efficiently and compactly (initial data suggests by a factor of 10-100×). Among other things, this may result in a smaller file size and lower computer memory and processing resources required to manipulate this data.

The fact that the mathematical framework is shared between different datasets may also allow for computational shortcuts during operations that require multiple datasets simultaneously (e.g. image registration or radiation dose calculation).

HSH representations of these datasets can also be incorporated into machine learning models such as artificial neural networks. The more compact representation of the data may be beneficial by simplifying the resulting neural network and leading to better use of training data.

Systems that could use the HSH representations of data for radiotherapy treatment planning and delivery include:
1) Image reconstruction algorithms that may be faster, less susceptible to artifacts, or provide additional information (also applicable to medical imaging in general),
2) Image registration algorithms that may be faster or more accurate,
3) Treatment plan optimization based on ultra-fast HSH dose calculation that leads to improved treatment plans.

### Example 2

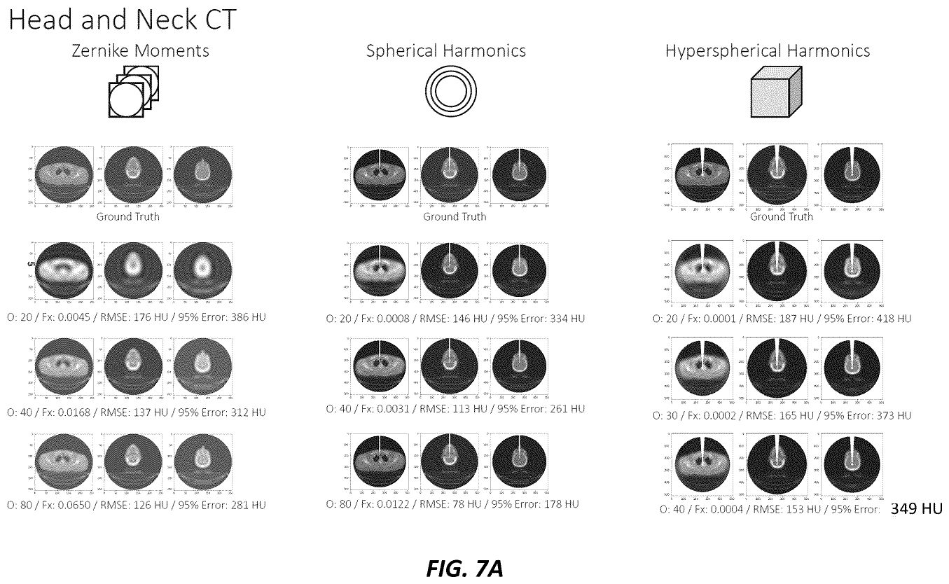

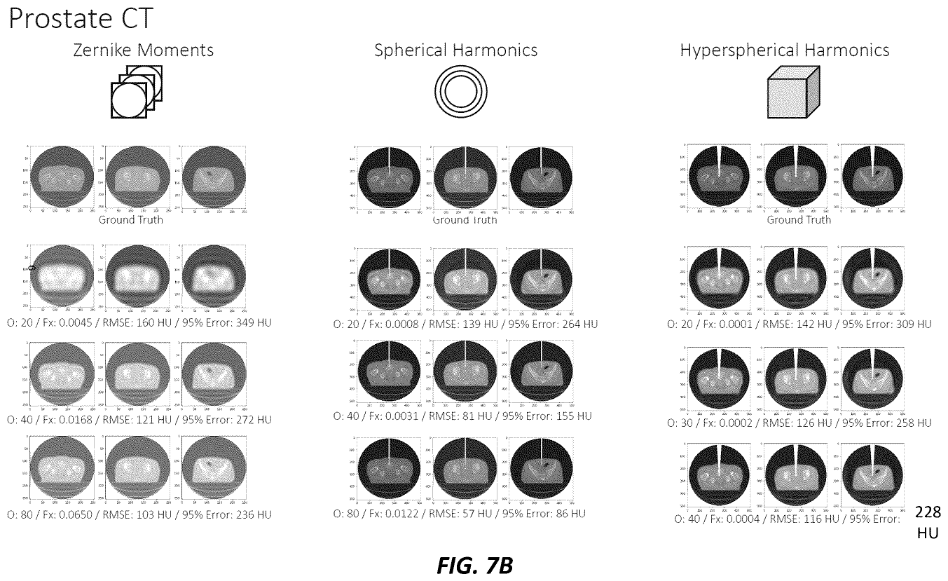

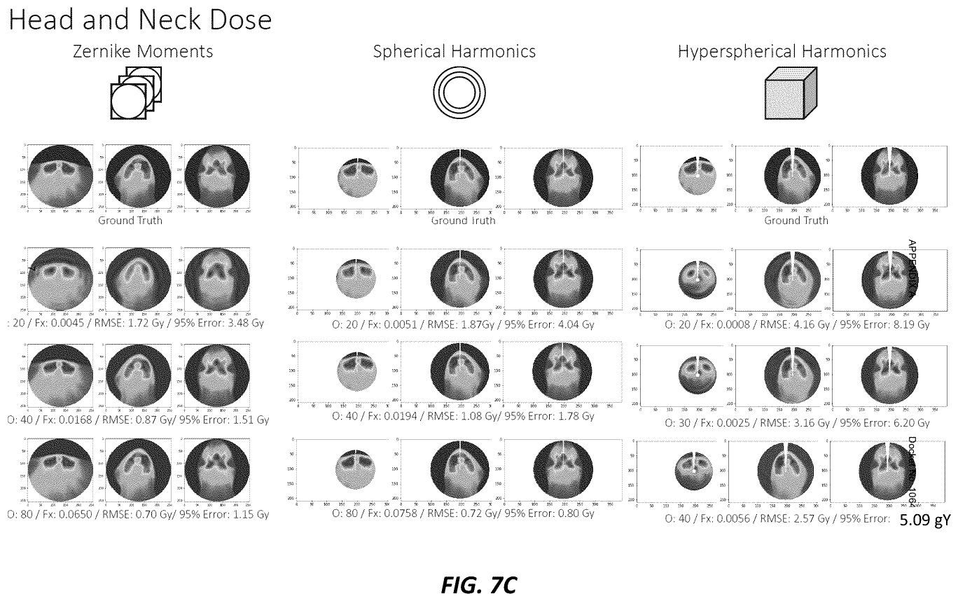

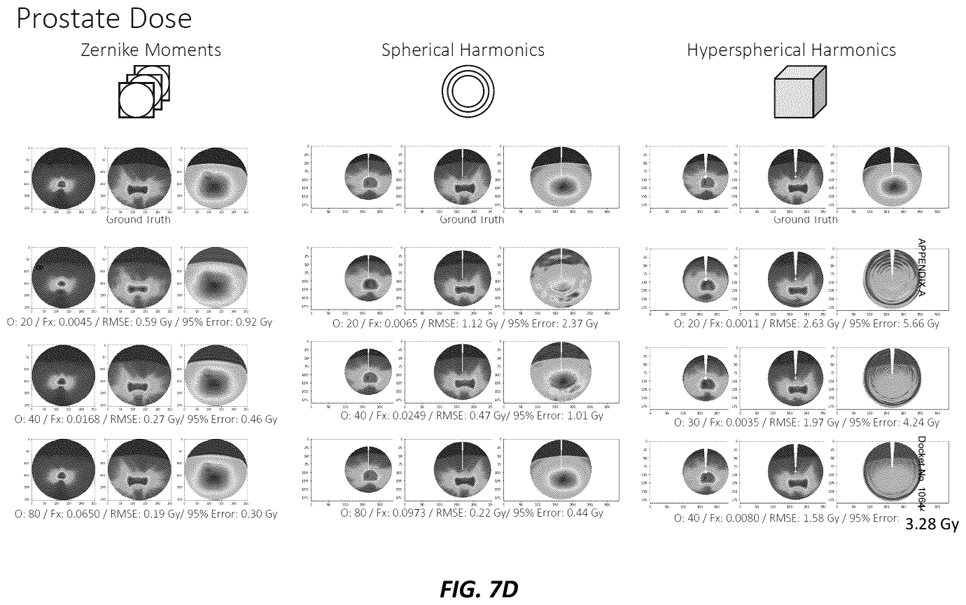

FIG. 7A-7D illustrates an example comparisons of three ways of representing the volumetric information of a CT scan and radiotherapy dose distribution for two patients: a head-and-neck cancer patient and a prostate cancer patient. FIG. 7A illustrates head and neck CT data, FIG. 7B illustrates Prostate CT data, FIG. 7C illustrates head and neck dose data, and FIG. 7D illustrates prostate dose data. The three ways are to model the volume using a weighted sum of basis functions comprised of either Zernike Moments, Spherical Harmonics, or Hyperspherical Harmonics. The domain of Zernike Moments is the 2D unit disk. Therefore, to cover the 3D volume of the data cubes, multiple sets of independent planar slices (each modeled with a series of Zernike Moments) must combined.

The domain of Spherical Harmonics is the 2D surface of a 3D sphere. Therefore, to cover the 3D volume of the data cubes, multiple sets of independent spherical shells (each modeled with a series of Spherical Harmonics) must be combined.

The domain of Hyperspherical Harmonics, however, is inherently 3D. Therefore, only a single set of Hyperspherical Harmonics is required to cover the 3D Volume of the data cubes. This is expected to result in the most compact representation of the data.

In each of FIGS. 7A-7D, the ground truth data being modeled is shown compared to images reconstructed from Zernike Moment, Spherical harmonic, and Hyperspherical Harmonic representations. Subsequent rows are labeled by the order of the basis function series used (O); the ratio of the total number of terms to the number of voxels in considered (Fx), which can be interpreted as a compression rate; the Root Mean Squared Error (RMSE) in units of Hounsfield Units (HU) for the CT data or Gray (Gy) for the dose data; and the 95th Percentile Error (95% Error) in units of Hounsfield Units (HU) for the CT data or Gray (Gy) for the dose data. The order of the set of basis functions used to approximate the data increases. With this change, the number of terms in the weighted sum increases, but so does the accuracy of the data representation.

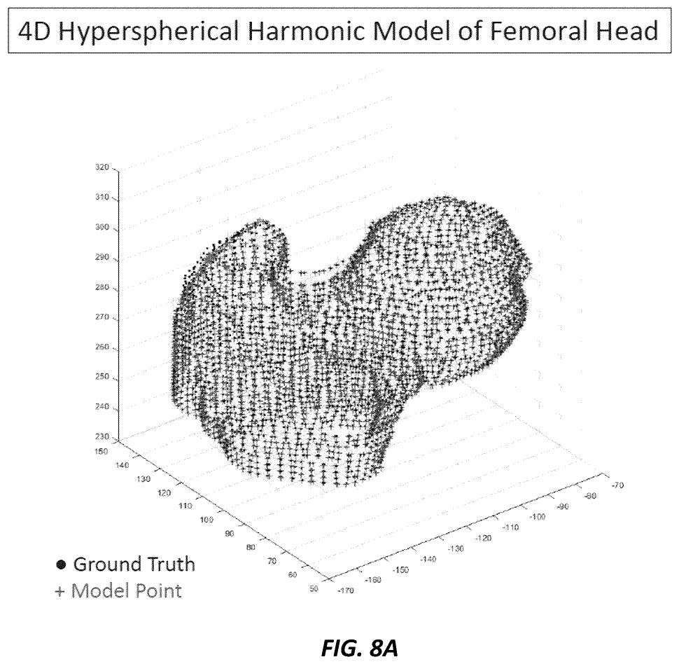

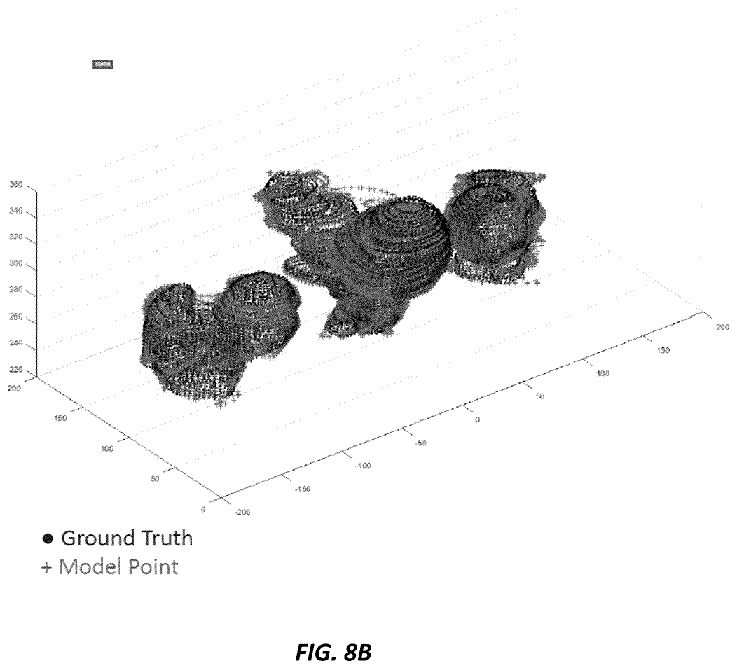

FIGS. 8A and 8B illustrate how 4D Hyperspherical Harmonics can be used to model additional anatomical structures. For example FIG. 8A illustrates a femoral head modeled as a point cloud. FIG. 8B illustrates a 4D Hyperspherical model of five anatomical structures simultaneously, including a prostate, bladder, rectum and both femoral heads, using a point cloud representation. The high dimensionality of Hyperspherical Harmonics allows for the modeling of not purely convex surfaces, and multiple surfaces structures simultaneously.

### Example 3

Implementations of the present disclosure include systems and methods adapted to radiotherapy planning for cancer patients. The current, resource-intensive process of radiotherapy (RT) planning poses a fundamental challenge to delivering the highest quality and safety of radiation to cancer patients. Creating a modern RT plan requires the optimization of a set of plan parameters from a complex, high-dimensional parameter space. The time required for computer systems to search this space and calculate the radiation dose of potential plans limits the ability of clinicians to identify an optimal plan that maximizes target dose and spares normal tissues. Therefore, clinicians often restrict the scope of the search and may conclude it prematurely, potentially compromising clinical outcomes. This is particularly salient for head-and-neck cancer patients (˜890,000 new cases per year, globally)). These patients require complex plans that take several days to create and feature multiple prescription dose levels and concave dose distributions. Finding parameters that satisfy the clinical objectives is a challenging, multi-factorial optimization problem. For simplification, the parameter space is typically restricted, for example, to a single treatment couch angle when additional angles can reveal a more optimal plan. The critical effect that dose calculation speed has on the resulting plan is emphasized by efforts to accelerate it. One strategy has been to predict plan doses using Deep Learning (DL) However, such DL models have complex network architectures that require high-volumes of training data, extensive computational resources, and subject expertise.

Furthermore, these models only apply to the specific treatment site and technique for which they were trained and are difficult to interpret. An alternative approach to rapid dose calculation without the limitations of DL models is required to better explore parameter space and identify more optimal plans. Otherwise, the RT planning process will remain resource-intensive and a primary source of quality and safety concerns.

Implementations of the present disclosure can be used to reduce or eliminate the intensive planning process as a logistical barrier to maximizing the quality and safety of RT plans. The example implementation can generate a simpler machine learning model for rapid dose calculation that avoids the challenges of DL approaches through the novel and strategic use of Hyperspherical Harmonics to represent RT data. Hyperspherical Harmonics can provide an effective framework with which to represent the complex data required to create RT plans, and machine learning models with these representations can be used for rapid dose calculation without the complexity of current DL models. As described throughout the present disclosure, Hyperspherical Harmonics (HSH) are the high-dimensional generalization of the mathematical spherical harmonic functions. HSH, however, has not been employed in RT. Here, HSH may have computational advantages because the geometries and symmetries of RT data closely align with HSH basis functions. This similarity suggests that HSH can represent RT data such as patient anatomy, radiation intensity maps, and dose distributions in a more compact form. An example implementation of the present disclosure shows that HSH representations of anatomy and dose required <1% of the original data size to achieve near-clinical accuracy.

Potential benefits of a compact HSH representation include: (i) decreased computational resources required for data manipulation, (ii) simpler machine learning models and better use of training data, (iii) the ability to integrate radiation physics principles into the models making them more interpretable, and (iv) a universal framework unrestricted by patient anatomy or treatment geometry. The present disclosure contemplates that such techniques can be applied not just to head and neck cancer patients, but for dose calculation in many other contexts, as well as in other imaging contexts that generate high dimensional data.

Two example benefits of the example implementation herein are:
Effective compression of RT data with HSH. The computer memory required to store RT data with clinical accuracy (error: <50 HU and <1 Gy) using HSH is <5% of that required with a conventional representation.
Enabling rapid dose calculation models using HSH representations of RT data. HSH representations of patient anatomy, radiation intensity maps, and dose distributions can be used in a simple machine learning model to perform dose calculation in <1 second.

These benefits enable additional plan options and treatment techniques, allowing clinicians to comprehensively compare clinical tradeoffs for personalized cancer care.

Modern RT plans feature complex patterns of small radiation beams directed towards the patient at many different angles. Optimizing the patient-specific set of plan parameters is critical for maximizing dose to the tumor (i.e. therapeutic benefit) while minimizing dose to nearby normal tissues (i.e. toxicity risk). However, the optimization space of possible plan parameters is large and complex. Computer systems require a significant amount of time to search this space and calculate the radiation dose of potential plans. As a result, clinicians will typically limit the search to a subset of the parameter space and conclude the search when a clinically-adequate plan is identified even if a more optimal plan exists. This resource-intensive process not only hinders the ability of clinicians to identify the optimal RT plan for an individual patient, but also limits the ability to broadly implement new advanced techniques.

While these planning challenges apply to every RT treatment site, their effects can be particularly salient when creating plans for head-and-neck cancer patients. Head-and-neck RT plans are some of the most complex. Intricate patterns of risk and disease progression lead to multiple prescription dose levels, and the close proximity of healthy organs to the disease requires convoluted dose distributions. Due to these planning challenges, head-and-neck RT plans are typically made by optimizing a single plan constrained to a single treatment couch angle using a pre-specified beam arrangement. The impact of this simplified approach is most clearly observed when compared to more comprehensive parameter searches. One advanced RT technique, 4πRT, extensively uses additional couch angles to achieve non-coplanar beams. This greater exploration of parameter space has been shown to improve the dosimetric plan quality of head-and-neck RT plans. For example, Rwigema et al. observed that 4πRT decreased dose to the parotid glands by 66% (decreasing the risk of toxicities like xerostomia) while simultaneously increasing the Tumor Control Probability by 3.7%. A second technique, Multi-Criteria Optimization (MCO), also features a more extensive search of possible plan parameters by generating not just one, but a series of candidate plans, each differently optimized. When presented with the MCO results, clinicians frequently preferred RT plans that would otherwise have not been considered, as the MCO plans improved all the normal tissue dose objectives by 0.6-3.2% on average. Lastly, online adaptive RT facilitated by Artificial Intelligence and Graphical Processing Units has the potential to improve plan doses by rapidly creating a new plan every day to match the changing patient anatomy. For example, online adaptive RT decreased the mean dose to the submandibular glands by 8 Gy and increased the minimum dose to the target by 0.5 Gy, thereby decreasing the probability of toxicity while also increasing the probability of disease control. The considerable benefits provided by these advanced techniques illustrate the compromises made during the conventional RT planning process.

A faster way to calculate radiation dose and evaluate the parameters of a candidate RT plan is required to facilitate a more extensive search of parameter space. Without this, i) the RT planning process will remain resource-intensive, ii) the broad implementation of advanced techniques will remain limited, and iii) the quality and safety of radiation delivered to cancer patients will remain compromised.

Three-Dimensional Spherical Harmonics (3D-SH) compose an orthonormal basis set whose domain is the 2D surface of a 3D sphere. Thus, they are well-suited to describe the surfaces of physical objects that are roughly spherical or that can be mapped onto a sphere. 3D-SH also require that the object be single-valued in spherical coordinates (i.e. “star-shaped”) which poses a challenge for objects that are multi-lobed or that feature holes or deep concavities.

For example, one technique models a set of bladder surface contours as a linear combination of 3D-SH. Because the geometry of 3D-SH was well-aligned with that of the bladder structures, the resulting description featured only 272 coefficients, which was much more compact than the original description composed of 1,596,375 voxels (0.02% compression ratio). 3D-SH was also used to model bladder surfaces. In this case, only 108 coefficients were required to achieve accuracy of within 1 mm when compared to the original description composed of 2,091 surface points (5.2% compression ratio). Another technique used 3D-SH to describe the notably non-star-shaped morphology of lumbar vertebrae by partitioning each vertebra into four sub-objects. Each sub-object was modeled with an individual set of 441 3D-SH coefficients, a decrease from the original 3,280 surface points (13.4% compression ratio). While partitioning the object into sub-objects is an effective method to work around the star-shape requirement of 3D-SH, it increases the overall size of the final shape descriptor.

Although the domain of 3D-SH is fundamentally 2D, efforts have been made to apply them to 3D volumetric data. One technique uses 3D-SH to describe a volumetric CT image by considering the data as a set of nested spherical shells. Each shell was, in turn, represented using 3D-SH, and the overall descriptor was composed of the coefficients of each shell. Another technique uses used 3D-SH to describe a volumetric distribution of radiation dose. Rather than considering the data as nested shells, the technique considers it as a stack of 2D slices that were each stereographically projected onto a sphere, which was then was represented using 3D-SH. The overall descriptor was composed of the coefficients of each slice. While these are two successful examples of extrapolating the use of the 3D-SH descriptor to 3D volumetric data, the advantages that make 3D-SH an appealing descriptor of 2D surfaces do not similarly extend to the higher dimensions. In both cases, the coefficients of each individual shell or slice are represented independently of the others. Correlations in the data between adjacent shells or slices are effectively ignored so cannot be used to compress the data further into a more compact representation.

Four-Dimensional Hyperspherical Harmonics (4D-HSH), as described herein, however, can be used to address the above limitations of 3D-SH. With the higher dimensionality, radial information can be considered simultaneously with angular data. This removes the requirement that objects must be star-shaped and allows 3D volumetric data to be modeled with a single set of coefficients. HSH can be used 4D-HSH to model multiple disconnected subcortical brain structures. Compared to 3D-SH representations, the higher-dimensional 4D-HSH representations were both more compact (420 vs. 5,292 coefficients) and more accurate (average mean squared error: 0.14 vs. 0.18). Such application of 4D-HSH has been limited to modeling surface point representations. Meanwhile, most RT data—including CT images, dose distributions, as well as other representations of anatomic structures—are instead represented by a 3D voxelized matrix.

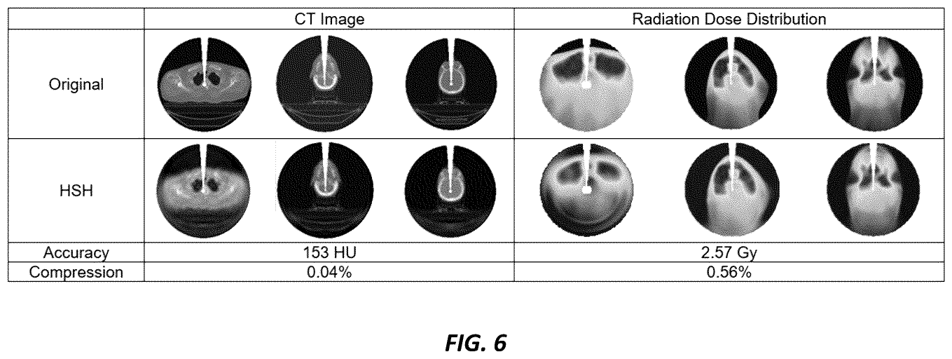

The present disclosure extends the use of 4D-HSH to voxelized data. For several example patients treated with RT for cancers of the head-and-neck or prostate, the present disclosure described the volumetric CT images and dose distributions each with a single set of 4D-HSH coefficients. Using 4D-HSH up to order 40, the example implementation was able to represent the CT images and the dose distributions with near-clinical accuracy (root mean square error: ≤153 HU and ≤2.57 Gy) using a very compact representation that was ≤0.8% the size of the original voxelized representation. These values are approaching clinical accuracy levels of ≤50 HU (Chourak et al. 2023) and ≤1 Gy (Mifton et al. 2018), and are expected to improve with HSH orders >40 while maintaining a small size. These data show that 4D-HSH can produce significantly more compact representations of the types of data that are used for RT planning. An example comparison of a CT Image and Radiation Dose distribution reproduced from the original image and the HSH representation are shown in FIG. 6.

Additionally, implementations of the present disclosure include improvements to rapid dose calculation models. Deep Learning (DL) methods have been applied to predict RT plan dose distributions. For example, one technique successfully used a 3D U-Net DL architecture to predict dose from collimated RT beams. The final model was fast, performing each individual inference in 0.6 seconds. It also accurately predicted the value of several dose objectives to within 1%. The technique is notable among the DL dose prediction literature for its input data. Typically, input data for dose prediction DL models includes descriptions of patient anatomy, such as their CT image and/or binary maps of structures. Instead, the technique generated spatial maps of five radiation physics parameters, including the distance of each measurement point to the radiation source and to the central beam axis. Using these parameters had the advantage of directly incorporating principles of radiation physics into the DL model.

However, having spatial maps of multiple parameters drastically increased the size and dimensionality of their input data. The example implementation of the present disclosure, described herein had compression ratios of ≤0.8%. This improvement will be even greater by a multiple when input data equivalent to the size of several CT images—five in one example technique—is required for the complex DL model architectures. In this way, using 4D-HSH representations according to implementations described herein is expected to reduce the size and dimensionality of input data and simplify the network architecture, thereby making more economical use of available training data. This applies whether the input data are CT images and binary structure maps, or if they are spatial maps of radiation physics parameters. In addition, the use of HSH can be extended to dimensions >4D to generate a single, compact representation of high-dimensional radiation physics parameters, such as the radiological depth to any anatomic point from any beam direction. This is expected to further decrease the input data size and dimensionality even more. The use of ≥4D-HSH as a compact representation of patient anatomy and radiation physics parameters is expected to play a critical role in simplifying the network architecture and training data requirements of rapid dose calculation models.

Implementations of the present disclosure can enable the mitigation of dose calculation as a primary quality-, safety-, and rate-limiting component of RT planning. Accordingly, implementations of the present disclosure provide clinicians with greater ability to explore parameter space and quickly identify more optimal RT plans for individual patients. The relaxation of restrictions that are typically applied when optimizing RT parameters can also unlock new treatment techniques that were previously impractical. Furthermore, rapid dose calculation facilitated by HSH representations of RT data can to disrupt the current clinical process and open new RT planning paradigms. Thus, implementations of the present disclosure enable high quality delivery of radiation to cancer patients by decreasing barriers to more optimal RT plans.

The status quo of RT planning uses pre-determined configurations of a restricted plan parameter optimization. Improved plans can result from a greater exploration of parameter space. Examples include techniques like 4πRT and MCO. Implementation of these techniques requires the creation and evaluation of numerous plans, which is currently limited by the time-intensive step of dose calculation.

The most accurate dose calculation strategies like Monte Carlo simulation or grid-based Boltzmann equation solvers simulate radiation transport through patient anatomy and require substantial amounts of time and computational resources. Alternatively, simplified dose calculation algorithms are faster but less accurate. Even though a single instance of these faster algorithms may not alone be prohibitively time-intensive, they are frequently repeated many times during the plan parameter optimization, and the cumulative effect is enough to dictate clinical workflows. Recent DL-based approaches attempt to predict a dose distribution based on descriptions of anatomy and beam parameters. The input to these models are large and high-dimensional, and in many cases, they are specific to the treatment site or technique.

The example implementation improves on conventional techniques, for example by its innovative representation of RT data with HSH and its novel and strategic use of these representations to create a simple rapid dose calculation model that can also directly incorporate radiation physics principles.

In addition to facilitating rapid dose calculations for RT plans, the example uses of HSH herein can enable new research, development, and application of RT. Unlike other representations of RT data, HSH provides a single, universally-applicable framework. Whether the data is an anatomic image, an anatomic structure, a dose distribution, or a radiation intensity map, all are expected to be well-characterized by HSH. Because HSH can be generalized to higher dimensions, they can also represent complex data like radiation physics parameters. Furthermore, the comprehensive geometry of HSH covers all possible RT treatment positions, angles, and configurations. Therefore, it can apply to techniques that use different geometries or technologies, likely including many not yet developed. The present disclosure enables HSH to be used as a universal and robust framework to determine the optimal relation between individual patient anatomy, RT plan technique, and treatment technology.

The computer memory required to store RT data with clinical accuracy (error: <50 HU and <1 Gy) using HSH can be <5% of that required with a conventional representation. An example implementation can represent the CT images, RT plans, and dose distributions of head-and-neck cancer patients in spherical coordinates and then transform them into a set of HSH basis function coefficients.

An example use case includes patients with a head-and-neck cancer diagnosis and a conventionally fractionated RT plan composed of 2 or 3 co-planar Volumetric Modulated Arc Therapy (VMAT) treatment fields. Each patient can have 2 or 3 treatment fields and each field can be considered a data point, so, for example, 50 patients corresponds to 100-150 data points. Therefore, implementations of the present disclosure can be used to generate training data for machine learning models. For each patient, the CT image, RT plan, and dose distributions can be exported from the clinical database and anonymized. Data can be securely transferred to an internal, pre-approved, HIPPA-compliant server for further manipulation.

Data Transformation and Analysis. Optionally, any or all of the CT images, RT plans, and dose distributions can be transformed into HSH representations. Each CT image along with the radiation intensity map and dose distribution from each treatment field can be converted into a spherical coordinate domain. The example implementation can also calculate the value of 4D-and 6D-HSH functions at the same coordinates. It can optimize a set of HSH coefficients to best represent the data using a fitting process such as least squares optimization. This process can be repeated using different numbers of HSH coefficients. The number of coefficients can be considered the size of the HSH representation. The size of the conventional representation will be the number of voxels within the spherical domain of the original data. The accuracy of each HSH representation can optionally be assessed by comparing it to the original data using metrics such as the root mean squared error (RMSE) and the 95^{th} percentile error.

As described throughout the present disclosure, the compact data representations described herein can have great implications on the computational resources required to store, manipulate, and operate on the data structures. The HSH representations can constitute the input and output feature vectors of a machine learning model, directly improving its dimensionality and reducing complexity.

In some implementations, compression ratios can be greater than 5% (e.g., 5-10% or 10-20%). The present disclosure contemplates that the number of HSH coefficients used can be adjusted to adjust the data representation size and accuracy for different use cases. Compression ratios of greater than 5% can therefore be of considerable practical benefit.

Implementations of the present disclosure further include rapid dose calculation models using HSH representations of RT data. Conventional dose calculation methods contribute to an RT planning process that is time-and resource-intensive, thereby limiting the quality and safety of radiation delivered to patients. Implementations of the present disclosure include a machine learning model that performs rapid dose calculation. HSH representations of patient anatomy, radiation intensity maps, and dose distributions can be used in a machine learning model to perform dose calculation in <1 second. An example implementation includes training an artificial neural network (ANN) that takes the HSH representations of patient anatomy and radiation intensity maps as input, and relates them to an HSH representation of the corresponding dose distribution as output. The trained machine learning model can expedite the planning process and allow greater exploration of plan parameter space. With this ability, clinicians can evaluate additional plan options and improved treatment techniques.

### Machine Learning

Model Design, Optimization, and Evaluation: The HSH representations described herein be used to create an ANN that relates the patient anatomy, radiation intensity map, and dose distribution. In an example implementation, two representations of patient anatomy are be considered. The first is a 4D-HSH representation of the original CT image. The second is a 6D-HSH map of radiological depth to any anatomic point from any beam direction. These HSH coefficients can be concatenated with the HSH coefficients of the radiation intensity map for an individual treatment field to form the input layer of the ANN. The output layer can be formed from the HSH coefficients of the corresponding dose distribution of the treatment field. The available data can be split into a training, validation, and testing data set in the ratio of 70:20:10. Model hyperparameters (e.g. number of hidden layers, number of nodes per layer, activation function, learning rate, and optimizer) can be varied to optimize the model performance on the validation data set.

Evaluation of Clinically Acceptable Results: The best performing model can be applied to the test data set to determine its efficacy. This can optionally be assessed in two ways. The first is to compare the true and predicted values of the HSH coefficients of the dose distribution in the test set. This can be the predictive performance of the model. The second can be to transform these predicted coefficients into a volumetric dose distribution and compare it with the true dose distribution. This can be the clinical relevance of the prediction. The two dose distributions can be compared using the RMSE of the voxel-by-voxel difference and also Gamma Analysis conducted at 3%/3 mm, 2%/2 mm, and 1%/1 mm with a 10% threshold (Low et al. 1998).

Dose Calculation Timing: Timing functions can be included in to measure the inference time of the trained machine learning models described herein. This time will be added to that required to transform the radiation intensity map into input HSH coefficients and that required to reconstruct the volumetric dose distribution from the output HSH coefficients. These steps are required to achieve a dose calculation process that starts from and ends with conventional data representations. The time required to transform the CT image into input HSH coefficients will be omitted as this can be precalculated once per patient regardless of the number of dose calculations. The mean duration of these steps measured across the test set will be considered the dose calculation time and compared to hypothesized threshold of 1 second.

The systems and methods described herein can enable rapid dose calculation with clinical accuracy by improving the training of machine learning models for dose calculation by pre-processing the medical data into HSH representations. As such, the example implementation can be used to drive plan parameter optimization and support treatment decisions. By using HSH representations of RT data as the input and output, the model can have a simpler architecture than current deep learning models. A simpler architecture can lessen the computational resources, time, and data required to create and implement the model, thereby increasing its applicability and facilitating its broader adoption. A rapid dose calculation model can lead to a greater exploration of plan parameter space, allowing clinicians to identify more optimal treatment plans and unlock new treatment techniques.

In sum, the implementations described herein can be used to provide a compact representation of RT data, enabling improvements to systems and methods for medical imaging and radiotherapy treatment. As explained herein, HSH can be used for sub-second dose calculation with clinical accuracy and other benefits within the radiotherapy and broader imaging fields. While the present disclosure is focused on the head and neck cancers, it should be understood that other treatment sites are possible, including the prostate and lung.

As yet another example application, the present disclosure contemplates creating a generative model of RT data and a computational pipeline using data from this model to calculate dose distributions with a Monte Carlo dose algorithm. The result can be an arbitrarily large set of synthetic, but realistic, training data made with extreme dose accuracy. A broadly-applicable dose calculation model can be created using the training data and used for RT plan optimization. In sum, the HSH-based methods described herein enable rapid creation of optimal RT plans regardless of disease site or treatment technique, removing the intensive planning process as a logistical barrier to maximizing the quality and safety of RT plans.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

What is claimed:

1. A computer-implemented method comprising:
determining a set of (n−1)-dimensional sample points in a spherical coordinate system;
calculating, at a respective coordinate of each of the set of (n−1)-dimensional sample points, respective values of individual n-dimensional Hyperspherical Harmonic (HSH) basis functions up through a given maximum order;
converting n-dimensional medical data into an HSH representation; and
constructing an approximation of the n-dimensional medical data from the HSH representation.

2. The computer-implemented method of claim 1, wherein converting the n-dimensional medical data into the HSH representation comprises representing the n-dimensional medical data using HSH by finding a set of coefficients that, when used to combine individual n-dimensional HSH basis functions in a weighted sum, minimize an error of a resulting value at each of the set of (n−1)-dimensional sample points compared to a true value of the n-dimensional medical data at each of the set of (n−1)-dimensional sample points.

3. The computer-implemented method of claim 1, wherein constructing the approximation of the n-dimensional medical data from the HSH representation comprises using the HSH representation as a series of coefficients to calculate a weighted sum of the respective values of individual n-dimensional HSH basis functions at each of the set of (n−1)-dimensional sample points, resulting in the approximation of the n-dimensional medical data.

4. The computer-implemented method of claim 1, wherein the n-dimensional medical data comprises a 4D Computed Tomography (CT) image, a 4D Magnetic Resonance Imaging (MRI) image, a 4D Positron Emission Tomography (PET) image, a 4D binary map of one or more anatomic structures, a 4D parametric map of one or more anatomic structures, a 4D radiation dose distribution from an external beam radiotherapy plan, a 4D radiation dose distribution from a brachytherapy plan, a 4D radiation dose distribution from one or more brachytherapy sources, a 5D multi-energy Computed Tomography (CT) image, a 5D multi-energy Cone-Beam Computed Tomography (CBCT) image, a 6D map of radiological depth through an image subject, a 4D radiation intensity distribution from a series of coplanar external beam radiotherapy plan radiation source positions, or a 5D radiation intensity distribution from a series of non-coplanar external beam radiotherapy plan radiation source positions.

5. The computer-implemented method of claim 1, further comprising estimating a 4D radiation dose distribution corresponding to a radiotherapy plan for a given anatomic configuration using the HSH representation of the n-dimensional medical data.

6. The computer-implemented method of claim 1, further comprising aligning the n-dimensional medical data by minimizing the value of a distance metric calculated between the HSH representation of the n-dimensional medical data and respective HSH representations of one or more different n-dimensional medical data sets.

7. The computer-implemented method of claim 1, further comprising estimating radiation intensity distribution corresponding to a radiation dose distribution for a given radiotherapy plan using the HSH representation of the n-dimensional medical data.

8. The computer-implemented method of claim 7, further comprising optimizing the given radiotherapy plan.

9. A computer-implemented method for performing a radiotherapy task comprising:
receiving a first HSH representation of first n-dimensional medical data;
receiving a second HSH representation of second n-dimensional medical data;
inputting the first HSH representation and the second HSH representation into a trained machine learning model; and
predicting, using the trained machine learning model, a third HSH representation of third n-dimensional medical data.

10. The computer-implemented method of claim 9, further comprising constructing an approximation of the third n-dimensional medical data from the third HSH representation.

11. The computer-implemented method of claim 9, further comprising converting the first n-dimensional medical data into the first HSH representation, and converting the second n-dimensional medical data into the second HSH representation.

12. The computer-implemented method of claim 9, wherein the radiotherapy task is calculating a radiation dose distribution that corresponds to a given anatomic configuration and radiation intensity distribution, the first n-dimensional medical data is an anatomic configuration as depicted in a medical image, the second n-dimensional medical data is a radiation intensity distribution, and the third n-dimensional medical data is a radiation dose distribution.

13. The computer-implemented method of claim 9, wherein the radiotherapy task is determining a set of boundaries of an anatomic structure, the first n-dimensional medical data is an anatomic configuration as depicted in a medical image, the second n-dimensional medical data is an indicator of an anatomic structure of interest, and the third n-dimensional medical data is a binary map or parametric map of the anatomic structure of interest.

14. The computer-implemented method of claim 9, wherein the radiotherapy task is reconstructing a medical image from a detected transmitted radiation signal, the first n-dimensional medical data is a radiation intensity distribution, the second n-dimensional medical data is a detected transmitted radiation signal, and the third n-dimensional medical data is an anatomic configuration as depicted in the medical image.

15. The computer-implemented method of claim 14, wherein the medical image is a Computed Tomography (CT) image, a Cone-Beam Computed Tomography (CBCT) image, a multi-energy Computed Tomography (CT) image, or a multi-energy Cone-Beam Computed Tomography (CBCT) image.

16. A system comprising:
a medical imaging device;
a server operably coupled to the medical imaging device, wherein the server is configured to:
receive a medical image from the medical imaging device;
convert the medical image into an hypespherical harmonic (HSH) representation by:
determining a set of (n−1)-dimensional sample points in a spherical coordinate system;
calculating, at a respective coordinate of each of the set of (n−1)-dimensional sample points, respective values of individual n-dimensional HSH basis functions up through a given maximum order;
converting n-dimensional medical data into an HSH representation; and
constructing an approximation of the n-dimensional medical data from the HSH representation;
a computing device in operable communication with the server by a network; and
a display operably coupled to the computing device; wherein the computing device is configured to:
retrieve, from the server, the HSH representation; and
output a depiction of the HSH representation for display to a user; and
in response to a user input, retrieve from the server, the medical image; and
output a depiction of the medical image for display.

17. The system of claim 16, wherein the computing device is further configured to: optimize a radiotherapy procedure based on the HSH representation.

18. The system of claim 16, wherein the computing device is further configured to receive a search query from the user, and, based on the search query, retrieve the HSH representation from the server.

19. The system of claim 16, wherein the medical imaging device comprises a computed tomography (CT) system.

20. The system of claim 16, wherein the server is further configured to receive a plurality of medical images from the medical imaging device, convert the plurality of medical images into a corresponding plurality of HSH representations, and index the plurality of HSH representations for search or retrieval of the corresponding plurality of medical images.
